(19) United States
(12) Patent Application Publication
(10) Pub. No.: US 2019/0314213 A1
(43) Pub. Date: Oct. 17, 2019

(54) TAMPONS COMPOSED OF AN INTEGRAL ABSORBENT MEMBER AND PROCESS FOR MAKING THE SAME

(71) Applicant: FIRST QUALITY HYGIENIC, INC., Great Neck, NY (US)

(72) Inventor: Christopher J. Graham, Lcok Haven, PA (US)

(21) Appl. No.: 16/385,235

(22) Filed: Apr. 16, 2019

Related U.S. Application Data
(60) Provisional application No. 62/658,700, filed on Apr. 17, 2018.

(51) Int. Cl. A61F 13/20; A61L 15/22; A61L 15/26; A61L 15/28; D04H 1/46

(52) U.S. Cl. CPC A61F 13/2054; A61F 13/2088; D04H 1/46; A61L 15/26; A61L 15/28; A61L 15/225

(57) ABSTRACT

An absorbent tampon made of a compressed strip of needle punched fiber having a needle punch density of at least 5 punches per cm². The strip has a minimum width of 30 mm and a minimum length of 100 mm and a minimum tensile strength of 10 N or greater.

### CROSS-REFERENCE TO RELATED APPLICATION

This application claims the benefit of U.S. Provisional Patent Application No. 62/658,700 filed Apr. 17, 2018, the contents of which are herein incorporated by reference in their entirety.

### FIELD OF THE INVENTION

This disclosure relates to absorbent tampons, and more specifically, relates to tampons with a high integrity absorbent member and processes for making such tampons.

### BACKGROUND

Commercial catamenial tampons have been available for decades and there are many different designs known in the art. Most designs include, at a minimum, an absorbent member and a withdrawal member.

The highest incidence risks associated with the use of catamenial tampons are those related to tampons shedding fibers, breaking, or otherwise disintegrating inside of the user's vagina. This loss of integrity is often the result of weakness or inconsistency in the construction of the absorbent fiber web and, therefore, the absorbent member. The loss of integrity may results in hazards, such as, for example, mild harms such as inconvenience and pain and serious harm such as pelvic inflammatory disease, a dangerous infectious condition. Users who experience tampons coming apart inside them often require medical attention. One common method in the art for mitigating this user risk is to include a thin fluid permeable overwrap or veil around the absorbent member to help contain the fibers. While this overwrap can help reduce shedding and increase overall tampon integrity, they are known to fail, especially on radially wound tampons.

In addition to the safety risks, low integrity absorbent members also create inefficiencies, scrap, and product performance variation in the tampon manufacturing process. This results from the absorbent members breaking or “necking down,” which causes machine stops and creates weight variations and defects. Weight variations lead to variation in tampon absorbency, dimensions, and integrity, all of which negatively impact a manufacturer's ability to produce products in a cost effective manner.

### SUMMARY OF THE INVENTION

There exists a need for absorbent members with improved integrity, particularly improved tensile strength, that can mitigate the risks of disintegrating tampons and that can improve conversion performance of manufacturing equipment. Ideally, such improved tampons are manufactured on manufacturing equipment that is easily modifiable and do not require significantly more absorbent fiber or greater dimensional configurations to attain desired absorbencies compared to current designs.

An object of the present invention is to provide catamenial tampons with improved absorbent member integrity and a method of manufacturing such tampons.

A tampon of this present invention can include configurations for all absorbencies defined by 21 CFR 801.430, including Light, Regular, Super, Super Plus, Ultra, and tampons with absorbency >18 g. A tampon according to an exemplary embodiment of the present invention is composed of an absorbent member comprising a mass of fibers entangled by needle punching having length direction and width direction tensile strengths of at least 10N when tested using the tensile method described herein. To form the absorbent member, a web of absorbent fibers is laid and entangled by needle punching at a needle punch density of at least 5 punches per cm². To form the tampon, the absorbent member is cut to length and consolidated into an uncompressed “blank.” The blank is then compressed to form the tampon pledget. With this tampon configuration and construction method, a tampon is created that has greater fiber integrity and, therefore, greater user safety and machine processability as compared to conventional tampons.

### BRIEF DESCRIPTION OF THE DRAWINGS

FIG. 1a is a representative diagram of a pledget without an overwrap according to an exemplary embodiment of the present invention;

FIG. 1b is a representative diagram of a pledget with an overwrap according to an exemplary embodiment of the present invention;

FIG. 2 is a profile view of an absorbent ribbon according to an exemplary embodiment of the present invention;

FIG. 3 shows a needle punching process according to an exemplary embodiment of the present invention;

FIG. 4 is a perspective view of radially rolled tampon according to an exemplary embodiment of the present invention;

FIG. 5 shows a pledget with applicator according to an exemplary embodiment of the present invention;

FIG. 6 is a flowchart showing steps of a fiber manufacturing process according to an exemplary embodiment of the present invention; and

FIG. 7 is a flowchart showing steps of a tampon manufacturing process according to an exemplary embodiment of the present invention.

### DETAILED DESCRIPTION OF THE INVENTION

The present invention is directed to an absorbent tampon composed of an absorbent member with improved integrity to prevent breakage, fiber loss, and otherwise disintegration, and a process for making the absorbent tampon. The present invention is not limited or restricted by the structures or particular configurations illustrated in the drawings.

As employed herein, the term “tampon” refers to any type of absorbent mass that is used in the vaginal cavity to absorb fluids such as menses. Tampons currently available in commerce are constructed of absorbent fibers known to be biocompatible, such as cotton and viscose rayon. Various tampon constructions are known, but most include the use of pads or strips of fiber mats (also known as fiber sliver, tape, or ribbon), termed “absorbent member” herein, that are compressed into a cylindrical or near-cylindrical shape. The compressed absorbent member along with any associated overwrap is termed “pledget” herein. It should be appreciated that in some contexts the terms tampon and pledget are interchangeable and that pledget can refer to both the pre-compressed and compressed forms. The term “tampon blank” is used herein to refer to the pre-compressed form.

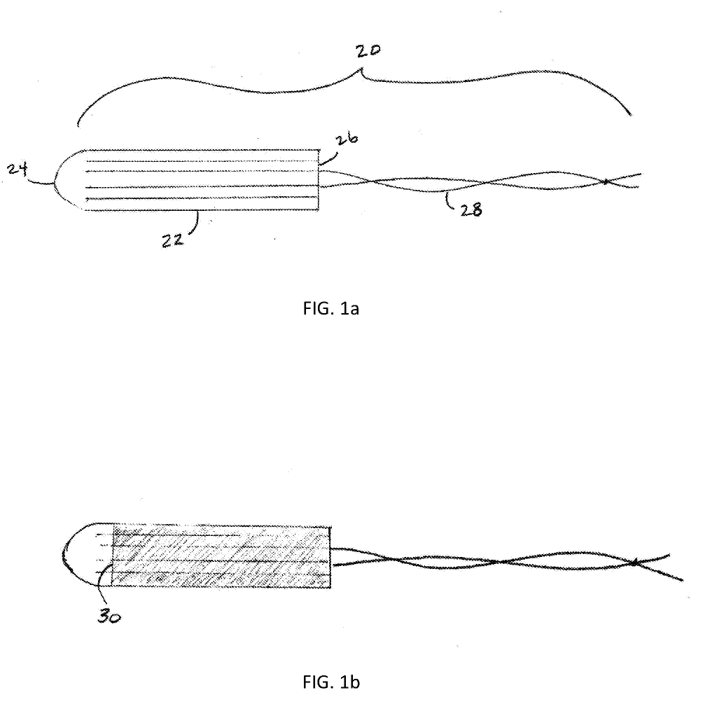

FIG. 1a shows a tampon, generally designated by reference number 20, according to an exemplary embodiment of the present invention. The tampon 20 is made up of a compressed absorbent member 22 and a withdrawal member 28. The absorbent member 22 has an insertion end 24 and a withdrawal end 26. The withdrawal member 28 is attached to or extends from the withdrawal end 26 of the absorbent member 22. The withdrawal member 28 may take the form of a cord or string. Various methods are known for attachment of withdrawal members to the tampon and include methods such as looping and knotting, sewing, punch through and looping, and others. In some embodiments, the withdrawal member is a cotton cord that is looped around the absorbent member and knotted prior to pledget formation.

FIG. 1b shows an exemplary embodiment where the absorbent member 22 is partly surrounded by a fluid pervious overwrap 30. The fluid pervious overwrap 30 can be an apertured or perforated film or a porous nonwoven. Nonwoven overwraps can be composed of natural or synthetic fibers or blends thereof. An example of a natural fiber suitable for use in the overwrap is cotton. Examples of synthetic fibers suitable for use in the overwrap include rayon and polyolefins. Various methods of nonwoven overwrap construction are known and include spunlacing, spunbonding and thermobonding. Apertured or perforated films can be composed of, for example, polyester, polyethylene, and/or polypropylene. In a preferred embodiment, the overwrap is a thermobonded nonwoven composed of polyethylene and polyester bicomponent fibers. In another embodiment, the overwrap is an apertured polyethylene film.

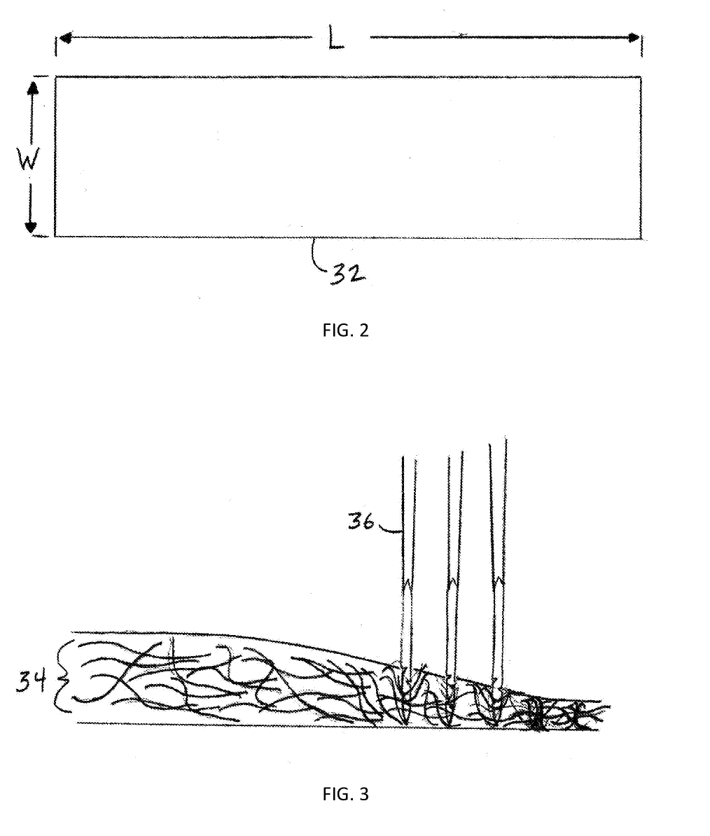

FIG. 2 shows an embodiment of an absorbent member 32, which is a strip of an absorbent fiber web that has been entangled by needle punching. The absorbent member 32 has a length L and a width W. In embodiments, the length and width of the absorbent member 32 is at least 100 mm and least 30 mm, respectively. In another embodiment, the length and width of the absorbent member 32 is at least 200 mm and at least 40 mm, respectively. In some embodiments, absorbent fibers used to form the absorbent member 32 include natural fibers, synthetic fibers, or blends thereof. In some embodiments, the average fiber sizes are at least 5 mm in length and preferably at least 20 mm in length. In some embodiments, the fibers can have various cross sectional shapes such as round, trilobal, flat, hollow, amorphous, and others. In embodiments, the absorbent member 32 is composed of cotton, rayon, or a blend of cotton or rayon where the average fiber sizes are at least 20 mm in length and the fiber cross-sections are generally round or trilobal.

In currently available tampons the absorbent member is created by forming webs of absorbent fibers that are entangled or bonded through chemical or mechanical means, and these absorbent members can be single or multilayer lamellar structures. FIG. 3 shows a needle punching process according to an exemplary embodiment of the present invention, which is a mechanical process for interlocking fibers. Fibers 34 are mechanically reoriented in the fiber web with needles 36, which results in the interlocking of the fibers 34. In embodiments, the absorbent member is made from a web of fibers that have been entangled by needle punching at a needle punch density of at least 5 punches per cm² and preferably at least 10 punches per cm².

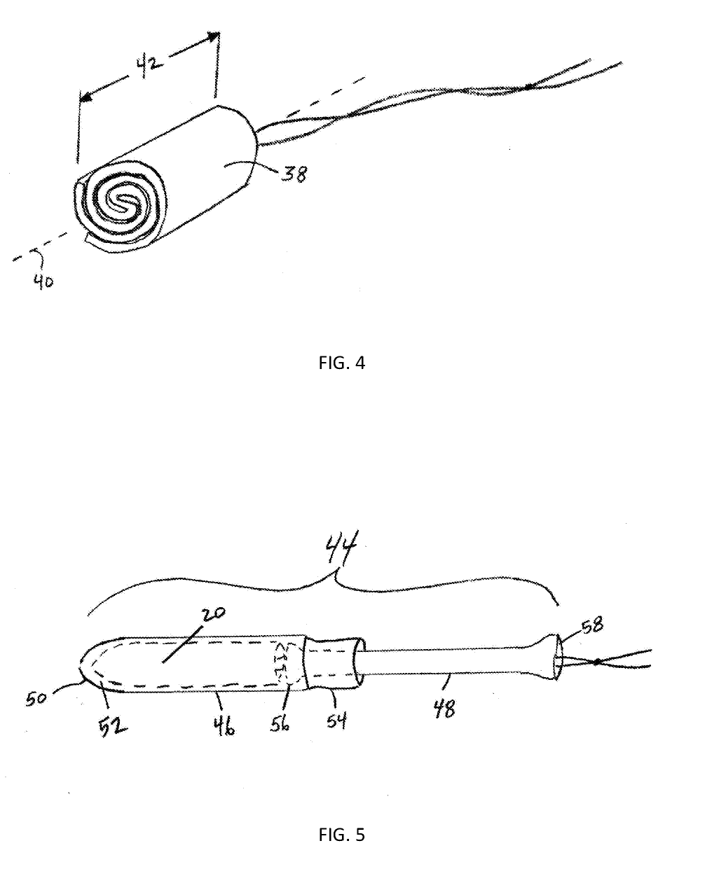

Several configurations of tampon blank construction from absorbent members are known, including simply compressing cut pads or further increasing fiber density before compression by rolling individual absorbent members or crisscrossing multiple absorbent members. FIG. 4 shows a tampon blank 38 according to an exemplary embodiment of the present invention. The tampon blank 38 is formed by coiling the absorbent member 32 radially around axis 40 into a substantially cylindrical shape with the height 42 of the cylinder being created by and equal to the width W of absorbent member 32. In an exemplary embodiment, the blank 38 is compressed into a substantially cylindrical pledget. In another exemplary embodiment, the blank 38 is layered in an accordion fashion and compressed into a cylindrical pledget. Other embodiments may include blanks that are compressed into wedges, blocks, cones, spheroids, or other conceivable shapes.

Tampons in accordance with the present invention are designed for insertion into the body by the user's finger, known as “digital” insertion, or with a tampon applicator. Tampon applicators are known, including those described in U.S. Pat. No. 4,921,474 to Suzuki and Masamitsu, the contents of which are incorporated herein by reference in their entirety.

FIG. 5 shows an applicator, generally designated by reference number 44, usable with a tampon according to an exemplary embodiment of the present invention. The tampon 20 is contained in applicator the 44. Applicator 44 has a barrel 46 and a plunger 48. Barrel 46 has an insertion end 50, a tampon housing chamber 52, and a finger grip 54. Plunger 48 has a tampon contacting end 56 and a finger contacting end 58. It should be appreciated that tampons in accordance with various exemplary embodiments of the present invention may be be used with any other known or later-discovered applicator.

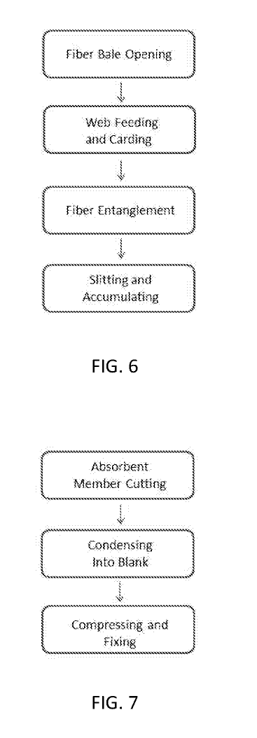

FIG. 6 is a flowchart showing a manufacturing process for constructing an absorbent member according to an exemplary embodiment of the present invention. The process includes fiber bale opening, web feeding, carding, fiber entanglement and slitting and accumulating. In another embodiment, additional layers are added to the web with additional inline cards to create a multi-layer web prior to entanglement. The multi-layer web allows for adjusting web basis weight to achieve the various tampon absorbencies. In another embodiment, the web is layered by cross-lapping to achieve the desired basis weight for the various tampon absorbencies. In another preferred embodiment, slit fiber is rolled into doffs for storage and conversion into tampons. In another preferred embodiment, slit fiber is festooned into containers for storage and subsequent conversion into tampons. Preferably, absorbent members are made by opening fiber, metering fiber into a web, carding the fiber, cross-lapping the web, needle punching to entangle, slitting, and rolling into rolls for storage and subsequent tampon conversion.

FIG. 7 is a flowchart showing a manufacturing process for constructing a tampon from an absorbent member according to an exemplary embodiment of the present invention. The process includes absorbent member cutting, condensing, and compressing and fixing. In a preferred embodiment an overwrap material is cut and added to the absorbent member prior to condensing into a tampon blank. In another preferred embodiment, the withdrawal member is attached prior to condensing. In another preferred embodiment, the tampon blank is compressed without the addition of process heat. In another preferred embodiment, the tampon blank compression is fixed with the addition of process heat of at least 100° C. to support shape sustainment post-compression. Process heat may be added during or after the initial compression step. In another preferred embodiment, microwave radiation is used during or after compression to support shape sustainment post-compression. Preferably, tampons are made by cutting an absorbent member, adding a nonwoven, looping a cord, condensing the absorbent member, compressing the absorbent member, and heating to set the compression.

The following test methods were used to demonstrate advantages of the present invention:

Tensile Strength

Sample preparation: To obtain the absorbent member, tampons were first unwrapped and removed from the applicator (if applicable). The overwrap, if present, was cut along the long axis of the tampon pledget. The absorbent member was then unrolled. To obtain standardized swatches for tensile pulls, two 30 mm×30 mm squares were cut from each absorbent member. Care was taken to ensure the square sides were approximately parallel to the length and width edges of the absorbent member.

Sample swatches were tested in an Instron pull force tester. To test, samples were clamped into 1 inch by 1 inch sets of square pneumatic jaws. The bottom set of jaws remained stationary and the top set of jaws was attached to a 500 N force cell. Prior to clamping the samples into the jaws, the top jaws were situated 15 mm from the bottom jaws. Once clamped in length-pull or width-pull orientations, samples were pulled vertically at a rate of 300 mm/min for 60 mm. Maximum load in Newtons (N) as defined as the peak of the stress/strain curve was recorded. Instron Bluehill software was used for recording and marking pull force data. Samples were tested in a climate controlled lab at room temperature.

The following Examples and Comparative Examples illustrate advantages of the present invention:

### EXAMPLE 1

A 144 gsm pad with dimensions of 150 mm×45 mm and an absorbency in the regular absorbency range was prepared using 100% viscose rayon fiber and needle punching at a punch density of 60 punches/cm².

### EXAMPLE 2

A 191 gsm pad with dimensions of 150 mm×50 mm and an absorbency in the super absorbency range was prepared using 100% viscose rayon fiber and needle punching at a punch density of 60 punches/cm².

### EXAMPLE 3

A 244 gsm pad with dimensions of 150 mm×50 mm and an absorbency in the plus absorbency range was prepared using 100% viscose rayon fiber and needle punching at a punch density of 60 punches/cm².

Comparative Example A: Store brand; 100% cotton radially wound tampon without overwrap; tampon is in an applicator tube.

Comparative Example B: U by K® Click®; 100% rayon radially wound tampon with overwrap; tampon is in an applicator tube.

Comparative Example C: o.b.; 100% rayon radially wound tampon with overwrap; packaged for digital insertion.

Absorbent members from Examples 1-3 and the Comparative Examples A-C were subjected to the tensile test method described above, and the results are shown in Table 1. Ten tensile strength measurements were taken from representative absorbent members, five in the length direction and five in the width direction. As shown in Table 1, in the on-market tampons, the absorbent member was considerably weaker in at least one of the two directions tested. Table 1 shows the lowest tensile strength of the two obtained measurements (length and width) for each of the absorbent members and demonstrates that the lowest tensile strength of the inventive absorbent member is substantially higher than that of all comparative tampons tested.

TABLE 1
minimum tensile pull value of the length and width pulls from each
absorbent member from Comparative Examples A-C and Examples 1-3 (N)

| | | | | | | | | | | EX. 1 | EX. 2 | EX. 3 |
|---|---|---|---|---|---|---|---|---|---|---|---|---|
| | COMP. EX. A | | | COMP. EX. B | | | COMP. EX. C | | | 144 | 191 | 244 |
| SAMPLE | Regular | Super | Plus | Regular | Super | Plus | Regular | Super | Plus | gsm | gsm | gsm |
| 1 | 1.718 | 2.33 | 4.493 | 1.221 | 1.366 | 2.143 | 2.143 | 1.445 | 1.370 | 43.65 | 55.71 | 52.41 |
| 2 | 1.554 | 2.833 | 2.610 | 1.890 | 2.543 | 2.748 | 2.610 | 1.810 | 1.350 | 30.51 | 51.80 | 62.08 |
| 3 | 1.447 | 2.80 | 1.806 | 1.754 | 1.584 | 3.978 | 1.806 | 1.108 | 1.393 | 23.45 | 49.62 | 60.60 |
| 4 | 1.228 | 3.051 | 2.202 | 1.464 | 2.915 | 1.923 | 1.923 | 1.149 | 1.537 | 39.13 | 53.90 | 56.31 |
| 5 | 1.991 | 3.632 | 2.181 | 1.666 | 1.08 | 3.066 | 2.181 | 2.265 | 1.213 | 29.64 | 44.95 | 65.04 |
| Largest | 1.991 | 3.632 | 4.493 | 1.890 | 2.915 | 3.978 | 2.610 | 2.265 | 1.537 | 43.65 | 55.71 | 65.04 |
| Minimum | | | | | | | | | | | | |
| Tensile | | | | | | | | | | | | |

The construction methods of the conventional tampon absorbent members cause the distribution of the tampon absorbent member's strength to be disproportionate between length and width directions. Ideally, a tampon absorbent member would be strong in both directions and the overall relationship between length and width tensile strengths would be similar. Processes in accordance with the present invention create a tampon absorbent member with substantially better relationship between tensile strength directions as compared to absorbent members formed using conventional processes. In this regard, Table 2 shows the average ratio of the maximum and minimum tensile directions for each absorbent member type, and specifically shows that Examples 1-3 have average ratios between unity and ≤2.00, while all Comparative Examples are greater than this.

TABLE 2
average ratio of maximum to minimum tensile strengths for absorbent
members from Comparative Examples A-C and Examples 1-3

| | Comp. Ex. A | | | Comp. Ex. B | | | Comp. Ex. C | | | | Ex. 1 | Ex. 2 | Ex. 3 |
|---|---|---|---|---|---|---|---|---|---|---|---|---|---|
| | Avg. | Avg. | Ratio | Avg. | Avg. | Ratio | Avg. | Avg. | Ratio | | Avg. | Avg. | Ratio |
| | Max | Min. | Max/ | Max | Min. | Max/ | Max | Min. | Max/ | Basis | Max. | Min. | Max/ |
| Absorbency | (N) | (N) | Min | (N) | (N) | Min | (N) | (N) | Min | Weight | (N) | (N) | Min |
| Regular | 5.164 | 1.588 | 3.252 | 17.798 | 1.599 | 11.131 | 9.970 | 1.373 | 7.264 | 144 gsm | 54.068 | 33.276 | 1.625 |
| Super | 10.355 | 2.929 | 3.535 | 20.736 | 1.898 | 10.927 | 9.688 | 1.555 | 6.229 | 191 gsm | 75.424 | 51.196 | 1.473 |
| Super Plus | 19.402 | 2.658 | 7.298 | 35.710 | 2.772 | 12.884 | 15.202 | 1.837 | 8.276 | 244 gsm | 104.038 | 59.288 | 1.755 |

While in the foregoing specification a detailed description of specific embodiments of the invention was set forth, it will be understood that many of the details herein given may be varied considerably by those skilled in the art without departing from the spirit and scope of the invention.

What is claimed is:

1. An absorbent tampon comprising:
a compressed strip of needle punched fibers having a needle punch density of at least 5 punches per cm², the strip having a width of at least 30 mm and a length of at least 100 mm, the absorbent tampon having a tensile strength of at least 10 N or greater.

2. The absorbent tampon of claim 1, wherein the fibers are natural fibers, synthetic fibers or blends of natural and synthetic fibers.

3. The absorbent tampon of claim 1, wherein the fibers are cotton, rayon or a blend of cotton and rayon fibers.

4. The absorbent tampon of claim 1, wherein the fibers are viscose rayon fibers.

5. The absorbent tampon of claim 1, wherein the fibers have an average fiber size of at least 5 mm in length.

6. The absorbent tampon of claim 1, wherein the fibers have an average fiber size of at least 20 mm in length.

7. The absorbent tampon of claim 1, wherein the fibers have a round, trilobal, flat, hollow or amorphous cross sectional shape.

8. The absorbent tampon of claim 1, wherein the needle punch density is 60 punches per cm².

9. The absorbent tampon of claim 1, further comprising a fluid pervious overwrap that at least partially surrounds the compressed strip of needle punched fibers.

10. The absorbent tampon of claim 9, wherein the overwrap is an apertured or perforated film.

11. The absorbent tampon of claim 9, wherein the overwrap is a porous nonwoven.

12. The absorbent tampon of claim 9, wherein the overwrap is made of natural fibers, synthetic fibers or blends of natural and synthetic fibers.

13. The absorbent tampon of claim 9, wherein the overwrap is made of a thermobonded nonwoven comprising polyethylene and polyester bicomponent fibers.

14. The absorbent tampon of claim 9, wherein the overwrap is made of an apertured polyethylene film.

15. An absorbent tampon comprising:
a compressed strip of needle punched fibers having a needle punch density of at least 5 punches per cm², the strip having a width of at least 30 mm and a length of at least 100 mm, the absorbent tampon having a maximum to minimum tensile ratio of about 2.0 or less.

16. A method of forming an absorbent tampon pledget, comprising:
needle punching a fiber web at a needle punch density of at least 5 punches per cm²;
cutting a strip from the fiber web so as to form a tampon blank, the strip having a width of at least 30 mm and a length of at least 100 mm; and
compressing the tampon blank into a shape so as to form the absorbent tampon pledget, the absorbent tampon having a tensile strength of at least 10 N or greater.

17. The method of claim 16, wherein the fiber web is made natural fibers, synthetic fibers or blends of natural and synthetic fibers.

18. The method of claim 16, wherein the fiber web is made of cotton, rayon or a blend of cotton and rayon fibers.

19. The method of claim 16, wherein the fiber web is made of viscose rayon fibers.

20. The method of claim 16, wherein the fiber web is made of fibers having an average fiber size of at least 5 mm in length.

21. The method of claim 16, wherein the needle punch density is 60 punches per cm².

22. The method of claim 16, further comprising the step of heating the pledget to a temperature of at least 100° C.

23. The method of claim 17, further comprising the step of exposing the pledget to microwave radiation.
